(19) United States
(12) Patent Application Publication
(10) Pub. No.: US 2025/0381054 A1
(43) Pub. Date: Dec. 18, 2025

(54) Ostomy Wafer Cover Device

(71) Applicant: Caroline Champagne, Parker, CO (US)

(72) Inventor: Caroline Champagne, Parker, CO (US)

(21) Appl. No.: 19/229,495

(22) Filed: Jun. 5, 2025

Related U.S. Application Data
(60) Provisional application No. 63/658,969, filed on Jun. 12, 2024.

(51) Int. Cl. A61F 5/44; A61F 5/443; A61F 5/445

(52) U.S. Cl. CPC A61F 5/4401; A61F 5/443; A61F 2005/4402; A61F 5/445

(57) ABSTRACT

An ostomy wafer cover device is disclosed, which is an ostomy wafer protection device that provides a sanitary, waterproof cover designed to protect the skin from maceration and the wafer from contamination during the removal of an ostomy pouch. The ostomy pouch cover device comprises a body component that is configured to cover an ostomy wafer. The body component includes a circular through hole which is sized and shaped to be positioned over the wafer of the ostomy pouch. The body component can have a slit or partial opening at the top to allow for easy insertion of the device onto the wafer and ostomy pouch. The body component also comprises adhesive to allow it to easily adhere to the ostomy pouch during use. Further, the body component is made of a flexible, waterproof material.

### CROSS-REFERENCE TO RELATED APPLICATION

The present application claims priority to, and the benefit of, U.S. Provisional Application No. 63/658,969, which was filed on Jun. 12, 2024, and is incorporated herein by reference in its entirety.

### FIELD OF THE INVENTION

The present invention relates generally to the field of ostomy wafer cover devices. More specifically, the present invention relates to an ostomy barrier attachment to prevent fecal spillage during ostomy pouch changes. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in ostomy wafer cover devices. Generally, fecal spillage on the skin barrier (i.e., wafer) during ostomy pouch changes is a common problem that necessitates replacement, a process that can be time-consuming and potentially irritating to the skin.

A surgical procedure known as a colostomy is performed on people in whom some portion of their large intestine has become inoperative due to disease or injury. A passageway, or stoma, is surgically formed on the patient through the abdominal wall, and the intestine is re-routed to the stoma. Waste material exits the body through this stoma, and a collection bag or appliance is attached to receive the waste material. Specifically, a skin barrier or wafer is the adhesive portion of the pouch or collection bag that is positioned around the user's stoma. It protects a user's skin and holds the pouch or collection bag in place. If feces spill on the wafer while changing the pouch, cleanup can be difficult, and in most cases, the wafer must be replaced. This is time-consuming and can irritate the skin.

Accordingly, there is a demand for an improved ostomy wafer cover device that provides a sanitary, waterproof cover designed to protect the skin from maceration and the wafer from contamination during the removal of an ostomy pouch. More particularly, there is a demand for an ostomy wafer cover device that is flexible and includes an adhesive to secure it in place during use.

Therefore, there exists a long-felt need in the art for an ostomy wafer cover device that provides users with a device that covers the barrier to prevent fecal spillage during ostomy pouch changes. There is also a long-felt need in the art for an ostomy wafer cover device that provides a sanitary, waterproof cover designed to protect the skin from maceration. Further, there is a long-felt need in the art for an ostomy wafer cover device that prevents contamination of the wafer during removal of an ostomy pouch. Moreover, there is a long-felt need in the art for a device that is flexible with an adhesive to secure it in place during use. Further, there is a long-felt need in the art for an ostomy wafer cover device that provides an elongated portion for cleanup of any overflow of the pouch. Finally, there is a long-felt need in the art for an ostomy wafer cover device that provides a waterproof cover to reduce the frequency of barrier replacements.

The subject matter disclosed and claimed herein, in one embodiment thereof, comprises an ostomy wafer cover device. The device is an ostomy wafer protection device that provides a sanitary, waterproof cover designed to protect the skin from maceration and the wafer from contamination during the removal of an ostomy pouch. The ostomy wafer cover device comprises of a body component that is configured to cover the pouching system that sticks to your body, aka ostomy wafer. The body component includes a circular through hole which is sized and shaped to be positioned under the flap of the wafer that is attached to the body. The body component can have a slit or partial opening at the top to allow for easy insertion of the device onto the wafer and ostomy pouch. The body component also comprises an adhesive to allow it to easily adhere under the ostomy pouch and around wafer flap during use. The body component is made of a flexible, waterproof material.

In this manner, the ostomy wafer cover device of the present invention accomplishes all of the foregoing objectives and provides users with a waterproof cover to protect the wafer from contamination. The device is a cover for the wafer of an ostomy pouch. The device is available in different sizes and shapes.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an ostomy wafer cover device. The device is an ostomy wafer protection device that provides a sanitary, waterproof cover designed to protect the skin from maceration and the wafer from contamination during the removal of an ostomy pouch. The ostomy pouch cover device comprises a body component that is configured to cover an ostomy wafer. The body component includes a circular through hole which is sized and shaped to be positioned over the wafer of the ostomy pouch. The body component can have a slit or partial opening at the top to allow for easy insertion of the device onto the wafer and under the ostomy pouch. The body component also comprises an adhesive to allow it to easily adhere to the ostomy wafer during use.

Typically, the ostomy wafer cover device is utilized to protect the skin from maceration and the wafer from contamination during the removal of an ostomy pouch, or other such pouch, such as colostomy bags, collection pouches for feces, urine, etc., or any other suitable medical product as is known in the art that a user wishes to remove without contamination. Generally, the term “ostomy” comprehends both colostomy and ileostomy (which involves the small intestine). In both conditions, collection pouches with wafers are used, and in both conditions, there are similar problems of wafer contamination during removal of the pouch. If feces spill on the wafer while changing the pouch, cleanup can be difficult, and in most cases, the wafer must be replaced. This is time-consuming and can irritate the skin. Draping the wafer under the ostomy pouch cover device before each change reduces the frequency of changes, reduces stress, and is more cost effective for the user than having to replace the wafer.

Generally, the ostomy wafer cover device is utilized with an ostomy bag and will be described with such, however, the device is not limited to use with only an ostomy bag and can be utilized with any suitable medical product as is known in the art, depending on the needs and/or wants of a user. Typically, the ostomy bag is made of a clear or transparent plastic film and has a skin barrier or wafer on its upper portion which is secured to a stoma on the patient's abdomen. A drain tube is provided at the bottom of the ostomy bag for withdrawing liquid or fluid wastes from the ostomy bag. The drain tube has a closure or stopper that can be removed to permit drainage of the ostomy bag.

In one embodiment, the ostomy wafer cover device comprises a body component. The body component is made from a single rectangle or square of material. The body component is sized and shaped to cover the entire front of an ostomy wafer and can be sized and shaped to also wrap over and around the sides of the wafer if desired to prevent any spills or messes while changing the pouch. The body component can be sized approximately six to twelve inches in length and approximately three to six inches in width to sufficiently cover the entire front of an ostomy wafer. The body component can be available in different sizes, depending on the size of the ostomy wafer it is to cover. For example, the ostomy wafer can be mini, midi, or maxi in size. A mini ostomy pouch is smaller and more discreet, often used for short periods or during activities like exercise. A midi ostomy pouch is medium in size and offers a balance between discretion and capacity. A maxi pouch is a larger pouch and provides more capacity, often used overnight or for those with higher output. The ostomy pouch is also available in different sizes for different sized patients, all the way from infant through small, medium, and large sizes for adults. Accordingly, the body component can be available in different sizes to fit any of these ostomy pouch sizes. Typically, the body component can be any suitable shape and size as is known in the art, as long as it is sized and shaped to cover the front side of an ostomy pouch.

In one embodiment, the body component comprises a through hole towards the top of the body component. Typically, the through hole is configured in a round shape and is sized to completely surround the wafer of the ostomy pouch but can be any suitable shape and size as long as the through hole is sized and shaped to fit securely around the perimeter of the wafer.

In one embodiment, the body component comprises a slit or partial opening positioned at the top of the body component. The slit starts at the top outer edge of the body component and extends down to the through hole. The slit allows a user to separate the body component at the slit, allowing access to the through hole, which allows a user to easily position the through hole around the wafer. Once in position, the opposing side edges of the slit overlap preventing any leaks from getting through to the ostomy pouch.

In one embodiment, the front perimeter surface of the through hole and the front surface of the slit comprises an adhesive, which allows the body component to be releasably secured to the ostomy pouch and removed as needed. Any suitable securing means can be utilized as is known in the art. In use, the wafer of the ostomy pouch would be inserted into the through hole of the body component and secured via the adhesive around the through hole to prevent contamination of the wafer. The adhesive at the slit, allows the overlapping edges of the slit to be sealed closed to prevent leaks.

In one embodiment, the body component comprises a front surface and a rear surface. The front surface is opaque, such that when positioned in front of the ostomy pouch, view of the ostomy wafer is blocked. Typically, the front surface is manufactured of an absorbent material, such as cotton. The front surface acts to absorb any spills, preventing any messes when changing the pouch. In one embodiment, the front surface of the body component comprises various colors, patterns and designs, and other aesthetic characteristics depending on the needs and/or wants of a user.

In one embodiment, the rear surface of the body component comprises an adhesive or adhesive portions, which allows the body component to be releasably secured to the ostomy pouch and removed as needed. Any suitable securing means can be utilized as is known in the art. In use, the body component is attached to the outside of the ostomy wafer. The rear surface is manufactured of a plastic material, such as polycarbonate, PVC, etc., to provide a waterproof barrier for the pouch. Thus, the body component provides a waterproof barrier between the waste and the wafer and makes it easy to wipe up any overflow quickly with the body component when changing the pouch.

In one embodiment, the ostomy wafer cover device is disposable. The body component is adhered to the ostomy pouch and then removed after use and disposed of. In one embodiment, the ostomy wafer cover device can be packaged and sold in packs of multiple units that can be used and disposed of, as needed. In another embodiment, the ostomy wafer cover device is packaged individually in sterile packages.

In yet another embodiment, the ostomy wafer cover device comprises a plurality of indicia.

In yet another embodiment, a method of protecting an ostomy wafer during removal of the ostomy pouch is disclosed. The method includes the steps of providing an ostomy wafer cover device comprising a body component with a through hole. The method also comprises positioning the through hole over a wafer of an ostomy pouch. Further, the method comprises securing the adhesive part of the body component over the wafer. The method also comprises securing the body component to the pouch. Finally, the method comprises changing the pouch while using the body component as a waterproof barrier to prevent contamination of the wafer.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the ostomy wafer cover device of the present invention showing the device and an ostomy wafer in accordance with the disclosed architecture;

FIG. 2 illustrates a perspective view of one embodiment of the ostomy wafer cover device of the present invention showing the device applied to a wafer in accordance with the disclosed architecture;

FIG. 3 illustrates a perspective view of one embodiment of the ostomy wafer device of the present invention showing the front surface of the device in accordance with the disclosed architecture;

FIG. 4 illustrates a perspective view of one embodiment of the ostomy wafer cover device of the present invention showing the front and back sides of the device in accordance with the disclosed architecture;

FIGS. 5-6 illustrate a perspective view of one embodiment of the ostomy wafer cover device of the present invention showing the front and back sides of different designs of the device in accordance with the disclosed architecture;

FIGS. 7A-C illustrate a perspective view of one embodiment of the ostomy pouch cover device of the present invention showing the front and back sides and the wafer inserted in accordance with the disclosed architecture;

FIGS. 8A-C illustrate a perspective view of one embodiment of the ostomy wafer cover device of the present invention showing different variations of the device in accordance with the disclosed architecture; and

FIG. 9 illustrates a flowchart showing the method of protecting an ostomy wafer during removal of the ostomy pouch in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for an ostomy pouch cover device that provides users with a device that covers the barrier to prevent fecal spillage during ostomy pouch changes. There is also a long-felt need in the art for an ostomy wafer device that provides a sanitary, waterproof cover designed to protect the skin from maceration. Further, there is a long-felt need in the art for an ostomy wafer cover device that prevents contamination of the wafer during removal of an ostomy pouch. Moreover, there is a long-felt need in the art for a device that is flexible with an adhesive to secure it in place during use. Further, there is a long-felt need in the art for an ostomy wafer cover device that provides an elongated portion for cleanup of any overflow of the pouch. Finally, there is a long-felt need in the art for an ostomy pouch cover device that provides a waterproof cover to reduce the frequency of barrier replacements.

The present invention, in one exemplary embodiment, is a novel ostomy wafer cover device. The ostomy wafer cover device comprises a body component that is configured to cover an ostomy wafer. The body component includes a circular through hole which is sized and shaped to be positioned over the wafer of the ostomy pouch. The body component can have a slit or partial opening at the top to allow for easy insertion of the device onto the wafer and under ostomy pouch. The body component also comprises an adhesive to allow it to easily adhere to the ostomy wafer during use. The body component is made of a flexible, waterproof material. The present invention also includes a novel method of protecting an ostomy wafer during removal of the ostomy pouch. The method includes the steps of providing an ostomy wafer cover device comprising a body component with a through hole. The method also comprises positioning the through hole over a wafer of an ostomy pouch. Further, the method comprises securing the adhesive part of the body component over the wafer. The method also comprises securing the body component to the pouch. Finally, the method comprises changing the pouch while using the body component as a waterproof barrier to prevent contamination of the wafer.

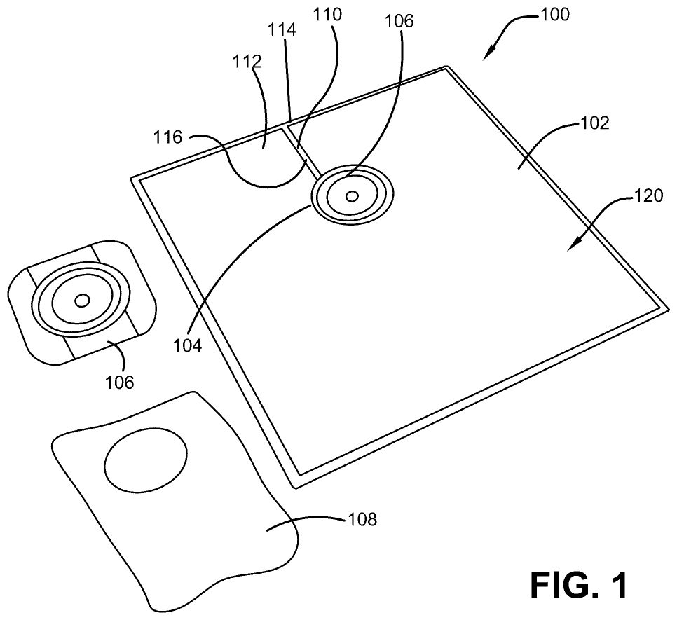

Referring initially to the drawings, FIG. 1 illustrates a perspective view of one embodiment of the ostomy wafer cover device 100 of the present invention. In the present embodiment, the ostomy wafer cover device 100 is an improved ostomy wafer cover device 100 that provides a user with a waterproof cover designed to protect the skin and prevent wafer contamination. Specifically, the ostomy wafer cover device 100 comprises a body component 102 with a through hole 104 which is sized and shaped to be positioned over the wafer 106 of the ostomy pouch 108. The body component 102 can have a slit 110 or partial opening at the top 112 to allow for easy insertion of the device 100 onto the wafer 106 and ostomy pouch 108.

Typically, the ostomy wafer cover device 100 is utilized to protect the skin from maceration and the wafer 106 from contamination during the removal of an ostomy pouch 108, or other such pouch, such as colostomy bags, collection pouches for urine, etc., or any other suitable medical product as is known in the art that a user wishes to remove without contamination. Generally, the term “ostomy” comprehends both colostomy and ileostomy (which involves the small intestine). In both conditions, collection pouches with wafers are used, and in both conditions, there are similar problems of wafer 106 contamination during removal of the pouch 108. If feces spill on the wafer 106 while changing the pouch 108, cleanup can be difficult, and in most cases the wafer 106 must be replaced. This is time-consuming and can irritate the skin. Draping the wafer 106 with the ostomy wafer cover device 100 before each change reduces the frequency of changes, reduces stress, and is more cost effective for the user than having to replace the wafer 106.

Generally, the ostomy wafer cover device 100 is utilized with an ostomy pouch 108 and will be described with such, however, the device 100 is not limited to use with only an ostomy pouch 108 and can be utilized with any suitable medical product as is known in the art, depending on the needs and/or wants of a user. Typically, the ostomy wafer 108 is made of a clear or transparent plastic film and has a skin barrier or wafer 106 on its upper portion which is secured to a stoma on the patient's abdomen. A drain tube is provided at the bottom of the ostomy pouch 108 for withdrawing liquid or fluid wastes from the ostomy pouch 108. The drain tube has a closure or stopper that can be removed to permit drainage of the ostomy pouch 108.

Further, the ostomy wafer cover device 100 comprises a body component 102. The body component 102 is made from a single rectangle or square of material. The body component 102 is sized and shaped to cover the entire front of an ostomy wafer 108 and can be sized and shaped to also wrap around the sides of the pouch 108 if desired to prevent any spills or messes while changing the pouch 108. The body component 102 can be sized approximately six to twelve inches in length and approximately three to six inches in width to sufficiently cover the entire front of an ostomy wafer 108. The body component 102 can be available in different sizes, depending on the size of the ostomy wafer 108 it is to cover. For example, the ostomy pouch 108 can be mini, midi, or maxi in size. A mini ostomy pouch is smaller and more discreet, often used for short periods or during activities like exercise. A midi ostomy pouch is medium in size and offers a balance between discretion and capacity. A maxi pouch is a larger pouch and provides more capacity, often used overnight or for those with higher output. The ostomy pouch 108 is also available in different sizes for different sized users, all the way from infant through small, medium, and large sizes for adults. Accordingly, the body component 102 can be available in different sizes to fit any of these ostomy wafer 108 sizes. Typically, the body component 102 can be any suitable shape and size as is known in the art, as long as it is sized and shaped to cover the front side of an ostomy wafer 108.

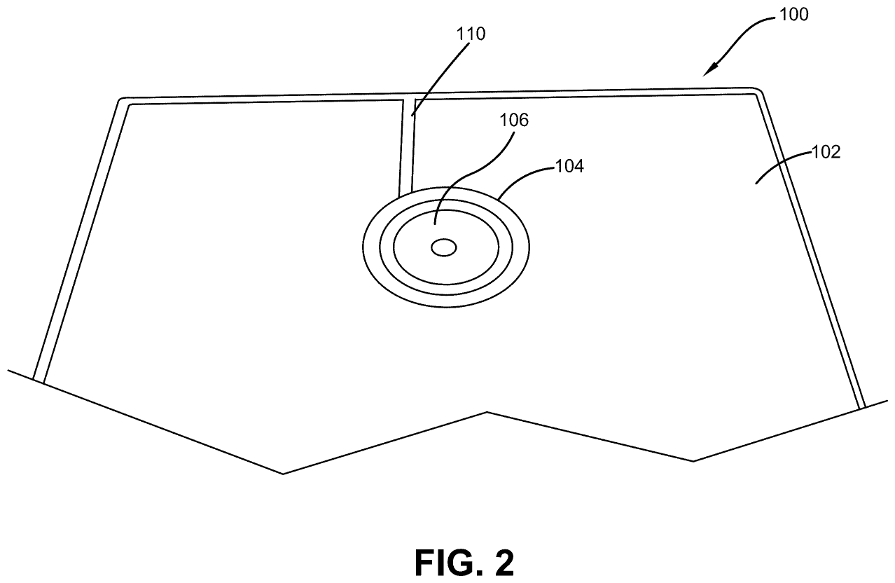

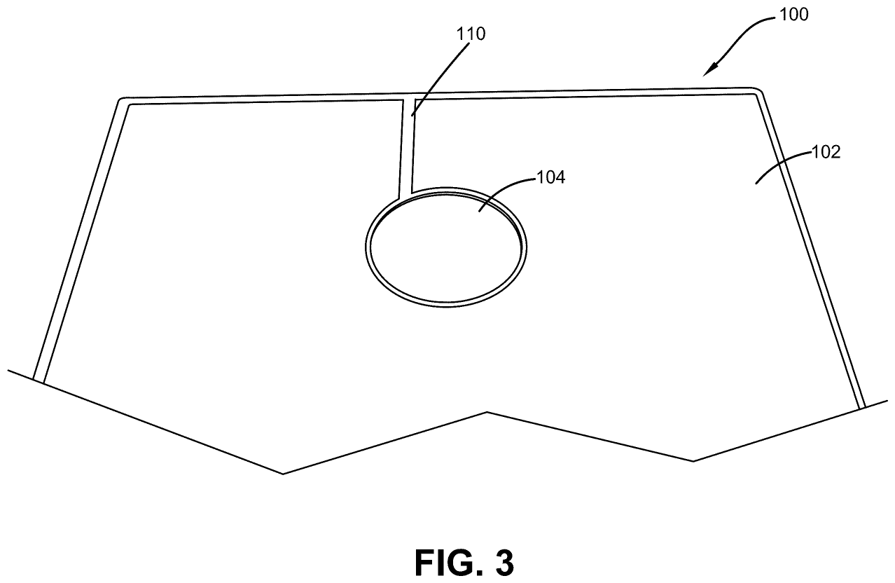

As shown in FIGS. 2-3, the body component 102 comprises a through hole 104 towards the top 112 of the body component 102. Typically, the through hole 104 is configured in a round shape and is sized to completely surround the wafer 106 of the ostomy pouch 108 but can be any suitable shape and size as long as the through hole 104 is sized and shaped to fit securely around the perimeter of the wafer 106.

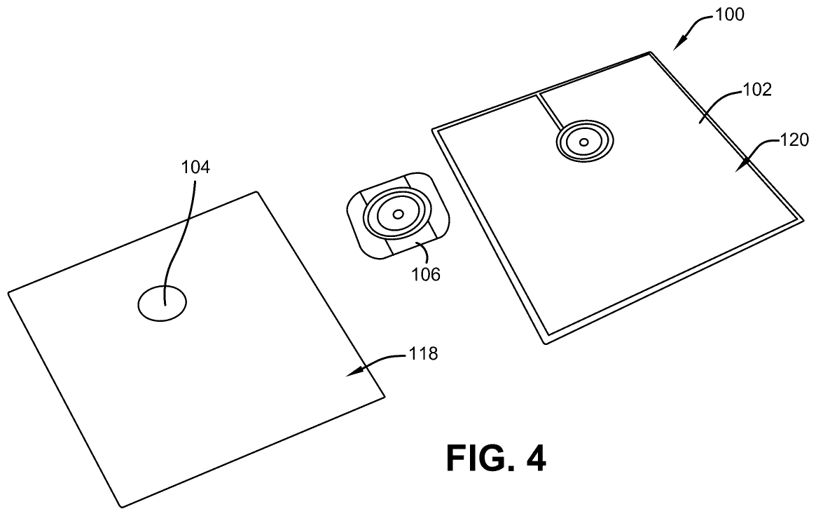

As shown in FIG. 4, the body component 102 comprises a slit 110 or partial opening positioned at the top 112 of the body component 102. The slit 110 starts at the top outer edge 114 of the body component 102 and extends down to the through hole 104. The slit 110 allows a user to separate the body component 102 at the slit 110, allowing access to the through hole 104, which allows a user to easily position the through hole 104 around the wafer 106. Once in position, the opposing side edges 116 of the slit 110 overlap preventing any leaks from getting through to the ostomy wafer 108.

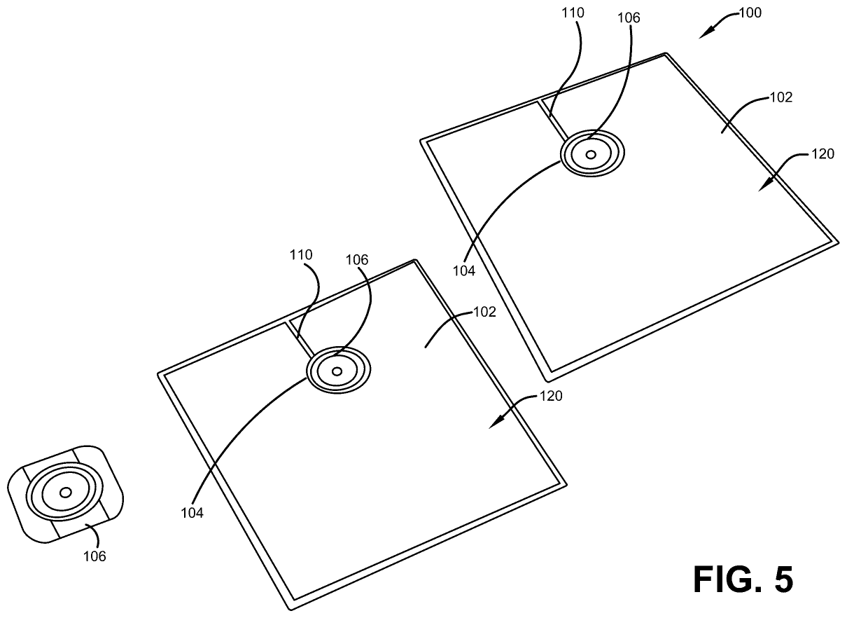

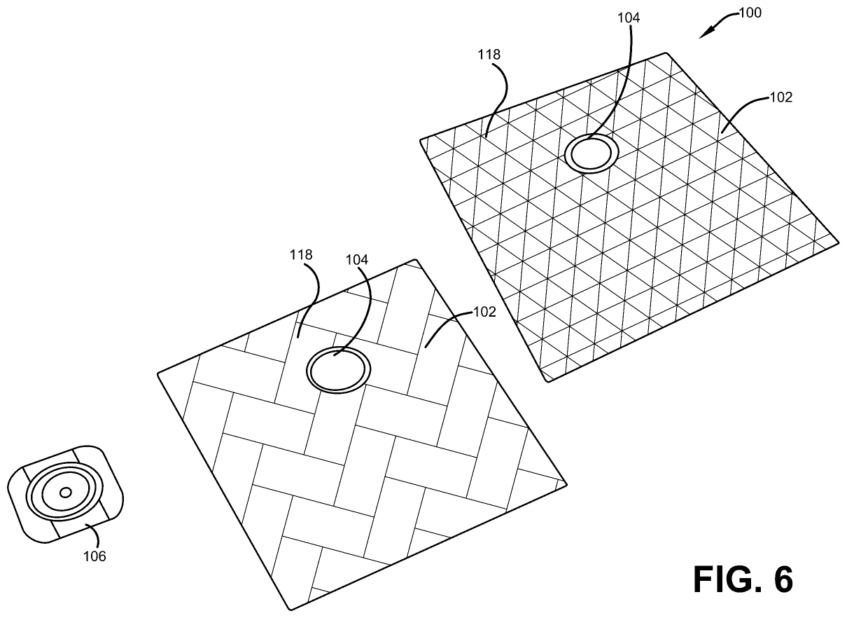

As shown in FIGS. 5-6, the body component 102 comprises a front surface 118 and a rear surface 120. The front surface 118 is opaque, such that when positioned in front of the ostomy wafer 108, view of the ostomy wafer 108 is blocked. Typically, the front surface 118 is manufactured of an absorbent material, such as cotton. The front surface 118 acts to absorb any spills, preventing any messes when changing the pouch 108. In one embodiment, the front surface 118 of the body component 102 comprises various colors, patterns and designs, and other aesthetic characteristics depending on the needs and/or wants of a user.

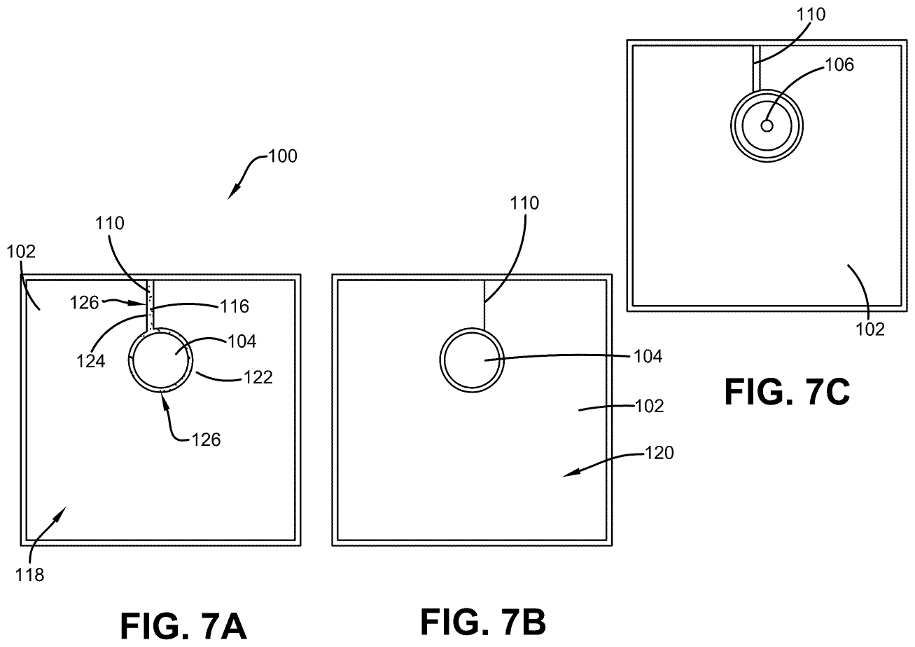

As shown in FIGS. 7A-C, the front perimeter surface 122 of the through hole 104 and the front surface 124 of the slit 110 comprises an adhesive 126, which allows the body component 102 to be releasably secured to the ostomy wafer 108 and removed as needed. Any suitable securing means can be utilized as is known in the art. In use, the wafer 106 of the ostomy pouch 108 would be inserted into the through hole 104 of the body component 102 and secured via the adhesive 126 around the through hole 104 to prevent contamination of the wafer 106. The adhesive 126 at the slit 110, allows the overlapping edges 116 of the slit 110 to be sealed closed to prevent leaks.

Further, in one embodiment, the rear surface 120 of the body component 102 comprises an adhesive 126 or adhesive portions, which allows the body component 102 to be releasably secured to the ostomy wafer 108 and removed as needed. Any suitable securing means can be utilized as is known in the art. In use, the body component 102 is attached to the outside of the ostomy wafer 108. The rear surface 120 is manufactured of a plastic material, such as polycarbonate, PVC, etc., to provide a waterproof barrier for the pouch 108. Thus, the body component 102 provides a waterproof barrier between the waste and the wafer 106, and makes it easy to wipe up any overflow quickly with the body component 102 when changing the pouch 108.

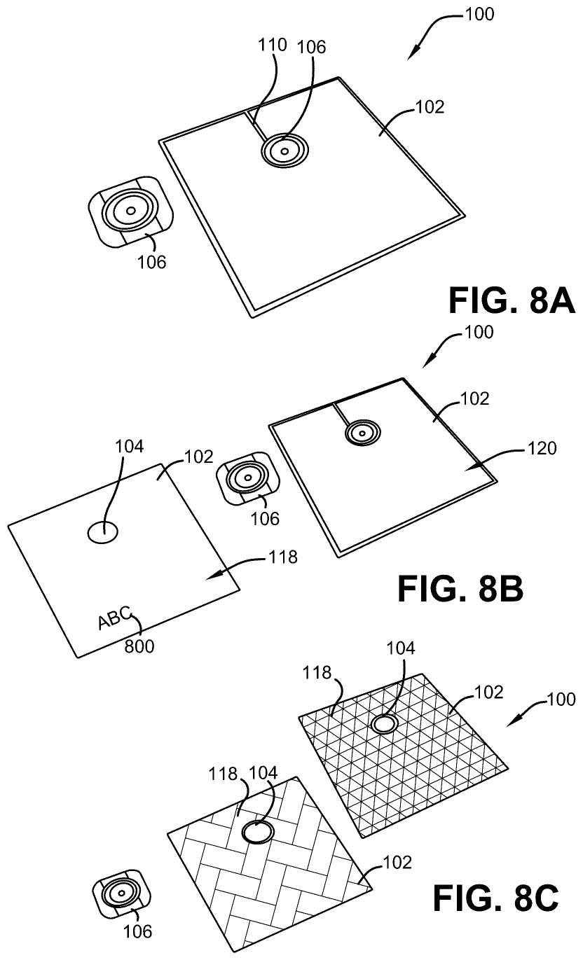

As shown in FIGS. 8A-C, in one embodiment, the ostomy wafer cover device 100 is disposable. The body component 102 is adhered to the ostomy wafer 108 and then removed after use and disposed of. In one embodiment, the ostomy wafer cover device 100 can be packaged and sold in packs of multiple units that can be used and disposed of, as needed. In another embodiment, the ostomy wafer cover device 100 is packaged individually in sterile packages.

In yet another embodiment, the ostomy pouch cover device 100 comprises a plurality of indicia 800. The body component 102 of the device 100 may include advertising, a trademark, or other letters, designs, or characters, printed, painted, stamped, or integrated into the body component 102, or any other indicia 800 as is known in the art. Specifically, any suitable indicia 800 as is known in the art can be included, such as, but not limited to, patterns, logos, emblems, images, symbols, designs, letters, words, characters, animals, advertisements, brands, etc., that may or may not be ostomy pouch, wafer, or brand related.

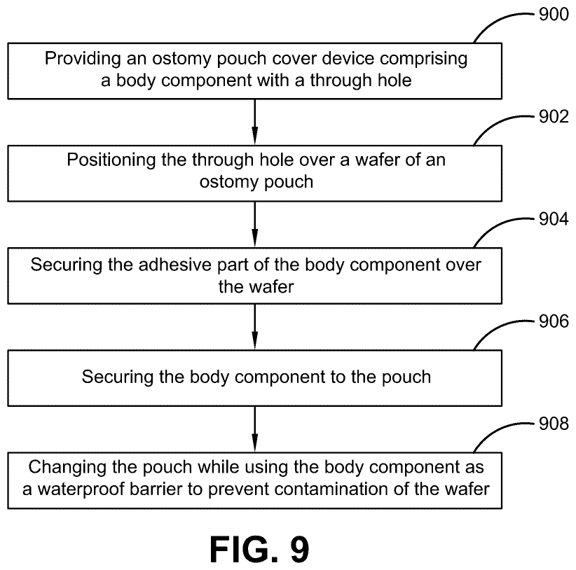

FIG. 9 illustrates a flowchart of the method of protecting an ostomy wafer during removal of the ostomy pouch. The method includes the steps of at 900, providing an ostomy pouch cover device comprising a body component with a through hole. The method also comprises at 902, positioning the through hole over a wafer of an ostomy pouch. Further, the method comprises at 904, securing the adhesive part of the body component over the wafer. The method also comprises at 906, securing the body component to the pouch. Finally, the method comprises at 908, changing the pouch while using the body component as a waterproof barrier to prevent contamination of the wafer.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “ostomy wafer cover device”, “ostomy pouch device”, “cover device”, and “device” are interchangeable and refer to the ostomy wafer cover device 100 of the present invention.

Notwithstanding the foregoing, the ostomy wafer cover device 100 of the present invention can be of any suitable size and configuration as is known in the art without affecting the overall concept of the invention, provided that it accomplishes the above-stated objectives. One of ordinary skill in the art will appreciate that the ostomy wafer cover device 100 as shown in FIGS. 1-9 is for illustrative purposes only, and that many other sizes and shapes of the ostomy wafer cover device 100 are well within the scope of the present disclosure. Although the dimensions of the ostomy pouch cover device 100 are important design parameters for user convenience, the ostomy wafer cover device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An ostomy wafer cover device that provides a user with a waterproof cover designed to prevent wafer contamination, the ostomy wafer cover device comprising:
a body component;
wherein the body component comprises a through hole which is positioned around a wafer of an ostomy pouch;
wherein once in position, the body component is sized and shaped to cover a top surface of the ostomy pouch; and
further wherein the body component protects a user's skin and prevents wafer contamination when changing the ostomy pouch.

2. The ostomy wafer cover device of claim 1, wherein the ostomy wafer cover device is utilized with an ostomy pouch that includes a transparent plastic film and a wafer on its upper portion which is secured to a stoma on a user's abdomen.

3. The ostomy wafer cover device of claim 2, wherein the body component is made from a single rectangle or square of material.

4. The ostomy wafer cover device of claim 3, wherein the body component is sized and shaped to cover an entire front of an ostomy wafer to prevent any spills or messes while changing the ostomy pouch.

5. The ostomy wafer cover device of claim 4, wherein the body component is sized approximately six to twelve inches in length and approximately three to six inches in width to sufficiently cover the entire front of an ostomy pouch.

6. The ostomy wafer cover device of claim 5, wherein the body component is available in different sizes.

7. The ostomy wafer cover device of claim 6, wherein the through hole is positioned towards a top of the body component.

8. The ostomy wafer cover device of claim 7, wherein the through hole is configured in a round shape and is sized to completely surround the wafer of the ostomy pouch.

9. The ostomy wafer cover device of claim 8, wherein the body component comprises a slit at the top of the body component for easy access to the through hole.

10. The ostomy wafer cover device of claim 9, wherein the slit starts at a top outer edge of the body component and extends down to the through hole, and allows a user to separate the body component at the slit, allowing access to the through hole.

11. The ostomy wafer cover device of claim 10, wherein opposing side edges of the slit overlap preventing any leaks from getting through to the ostomy pouch.

12. The ostomy wafer cover device of claim 11, wherein the body component comprises a front surface and a rear surface.

13. The ostomy wafer cover device of claim 12, wherein the front surface is opaque and manufactured of absorbent material.

14. The ostomy wafer cover device of claim 13, wherein the front perimeter surface of the through hole and the front surface of the slit comprises an adhesive, which allows the body component to be releasably secured to the ostomy wafer and removed as needed.

15. An ostomy wafer cover device that provides a user with a waterproof cover designed to prevent wafer contamination, the ostomy pouch cover device comprising:
a body component comprises a front surface and a rear surface;
wherein the body component is sized and shaped to cover an entire front of an ostomy wafer to prevent any spills or messes while changing the ostomy pouch;
wherein the body component comprises a through hole which is configured in a round shape and is sized to completely surround a wafer of the ostomy pouch;
wherein a front perimeter surface of the through hole comprises an adhesive, which allows the body component to be releasably secured to the wafer and removed as needed;
wherein the body component comprises a slit at the top of the body component for easy access to the through hole;
wherein opposing side edges of the slit overlap and are sealed via adhesive, preventing any leaks from getting through to the ostomy pouch;
wherein the front surface is opaque and manufactured of absorbent material;
wherein the rear surface is manufactured of a waterproof material to create a waterproof barrier; and
further wherein the body component protects a user's skin and prevents wafer contamination when changing the ostomy pouch.

16. The ostomy wafer cover device of claim 15, wherein the rear surface of the body component comprises an adhesive or adhesive portions, which allows the body component to be releasably secured to the ostomy pouch and removed as needed.

17. The ostomy wafer cover device of claim 15, wherein the ostomy pouch cover device is disposable.

18. The ostomy wafer cover device of claim 15, wherein the body component comprises various colors, patterns and designs.

19. The ostomy wafer cover device of claim 15 further comprising a plurality of indicia.

20. A method of protecting an ostomy wafer during removal of the ostomy pouch, the method comprising the following steps:
providing an ostomy wafer cover device comprising a body component with a through hole; positioning the through hole over a wafer of an ostomy pouch;
securing the adhesive part of the body component over the wafer;
securing the body component to the pouch; and
changing the pouch while using the body component as a waterproof barrier to prevent contamination of the wafer.
